(19) United States
(12) Patent Application Publication
(10) Pub. No.: US 2026/0018286 A1
(43) Pub. Date: Jan. 15, 2026

(54) PREDICTING PATIENT OUTCOMES RELATED TO CANCER

(71) Applicant: Valar Labs, Inc., Palo Alto, CA (US)

(72) Inventors: Anirudh Rajiv Joshi, Palo Alto, CA (US); Damir Vrabac, Palo Alto, CA (US); Viswesh Krishna, Cupertino, CA (US); Vrishab Krishna, Cupertino, CA (US); Vivek Uday Nimgaonkar, Baltimore, MD (US)

(21) Appl. No.: 18/849,062

(22) PCT Filed: Nov. 7, 2023
(86) PCT No.: PCT/US2023/078973

§ 371 Date: Sep. 20, 2024

Related U.S. Application Data
(60) Provisional application No. 63/424,855, filed on Nov. 11, 2022.

(51) Int. Cl. G16H 50/20; G06V 10/764; G06V 10/82; G06V 20/69; G16H 10/60; G16H 30/40; G16H 50/30

(52) U.S. Cl. CPC G16H 50/20; G06V 10/764; G06V 10/82; G06V 20/698; G16H 10/60; G16H 30/40; G16H 50/30; G06V 2201/03

(57) ABSTRACT

Disclosed are systems and methods for an artificial intelligence based pathology platform that can provide prognostic value to clinicians. For example, the platform can predict outcomes related to a cancer, and may include the steps of obtaining a histological sample of a cancer tumor of a patient, determining a feature set for the histological sample by applying a deep learning module trained on a population of histological samples of cancer tumors of the same type as the obtained histological sample of the cancer tumor, and generating an outcome set for the patient by applying a second model to the determined feature set.

### CROSS-REFERENCE TO RELATED APPLICATIONS

This application claims the benefit of priority from U.S. Provisional Application No. 63/424,855, filed on Nov. 11, 2022, and entitled, “Predicting Patient Outcomes Related to Cancer,” the contents of which are hereby fully incorporated by reference.

### TECHNICAL FIELD

The present disclosure relates to predicting patient outcomes related to cancer, e.g., using machine learning models.

### BACKGROUND

Cancer is a leading cause of death worldwide, and accounts for close to one in six deaths. However, many cancers can be cured if treated effectively and early. Pathological samples from a cancer patient are often analyzed for clinical metrics of risk and may be used to determine the most appropriate treatment for that patient. However, conventional methods that estimate clinical metrics of risk are often limited in design and may overestimate or underestimate the risk of progression and/or recurrence of cancer within a patient.

### SUMMARY

Embodiments of the present disclosure include techniques for applying a machine learning model to pathology samples to predict outcomes, stratify risk, and predict responses to cancer therapy.

In some embodiments, a method performed by at least one processor for predicting outcomes related to a cancer, may include the steps of obtaining a histological sample of a cancer tumor of a patient, determining a feature set for the histological sample by applying a deep learning module trained on a population of histological samples of cancer tumors of the same type as the obtained histological sample of the cancer tumor, and generating an outcome set for the patient by applying a second model to the determined feature set. Optionally, the outcome set may include at least one of a risk category, or risk-score for at least one of recurrence free survival, progression free survival, event free survival, overall survival, response to therapy, or disease-free survival. The cancer may be at least one of bladder cancer, non-muscle invasive bladder cancer, muscle invasive bladder cancer, urothelial carcinoma of the bladder, squamous cell carcinoma of the bladder, adenocarcinoma of the bladder, and small cell carcinoma of the bladder. The method may also include the step of providing a set of recommended therapies responsive to the determined feature set for the histological sample. Optionally, the feature set for the histological sample may include at least one of morphology data, tissue region data, spatial relationship data, colocalization data, and hotspot data. In some embodiments, the deep learning module includes a U-Net model, where the U-Net model comprises a fully convolutional neural network having an encoder and decoder. In some embodiments, the deep learning module is trained on the population of histological samples of cancer tumors of the same type as the obtained histological sample of the cancer tumor to determine nuclei location and shape data. In some embodiments, determining a feature set for the histological sample further includes the steps of determining locations of tissue within the histological sample, detecting positions of nuclei and cells of interest within the determined locations of tissue, determining at least one of morphologic, geometric, and textural features for each of the detected nuclei and cells of interest, and determining a spatial location feature for each of the detected nuclei and cells of interest. Optionally, the second model may include a multivariate model. In some embodiments, the multivariate model includes a Cox proportional hazards (CPH) model. In some embodiments, training the second model on non-histological data includes at least one of medical images, clinical variables, genomics, and medical text. Further, the second model may be trained to determine a signature, wherein the signature comprises the combination of histological features and weights. In some embodiments, the method includes the step of administering to the patient the particular treatment type, responsive to the outcome set for a particular treatment type. Further, the method may also include the step of displaying, on a graphical user interface, at least a portion of the outcome set.

In some embodiments, a non-transitory computer-readable medium may store instructions that, when executed on one or more processors, cause the one or more processors to obtain a histological sample of a cancer tumor of a patient, determine a feature set for the histological sample by applying a deep learning module trained on a population of histological samples of cancer tumors of the same type as the obtained histological sample of the cancer tumor, and generate an outcome set for the patient by applying a second model to the determined feature set. Optionally, the instructions may also cause the one or more processors to display, on a graphical user interface, at least a portion of the outcome set. Optionally, the instructions may also cause the one or more processors to determine the feature set for the histological sample by determining locations of tissue within the histological sample, detecting positions of nuclei and cells of interest within the determined locations of tissue, determining at least one of morphologic, geometric, and textural features for each of the detected nuclei and cells of interest, or determining a spatial location feature for each of the detected nuclei and cells of interest. In some embodiments the second model includes a multivariate model.

In some embodiments, a system for predicting outcomes related to a cancer, includes at least one server communicatively coupled to a user device by a network, wherein the at least one server further comprises a non-transitory memory storing computer-readable instructions and at least one processor. The execution of the computer-readable instructions causing the at least one server to train a deep learning module on a population of histological samples of cancer tumors, wherein the deep learning module comprises a U-net model, train a second model on feature set data and outcomes data, wherein the second model comprises a multivariate model, obtain a histological sample of a cancer tumor of a patient, wherein the cancer tumor is of the same type as the population of histological samples of cancer tumors, determine a feature set for the histological sample by applying the trained deep learning module, and generate an outcome set for the patient by applying the trained multivariate model to the determined feature set.

In some embodiments, the feature set includes at least one of morphology data, tissue region data, spatial relationship data, colocalization data, and hotspot data. Optionally, determining the feature set may include the execution of computer-readable instructions causing the at least one server to: determine locations of tissue within the histological sample, detect positions of nuclei and cells of interest within the determined locations of tissue, determine at least one of morphologic, geometric, and textural features for each of the detected nuclei and cells of interest, or determine a spatial location feature for each of the detected nuclei and cells of interest. In some embodiments, the outcome set includes at least one of a risk category, or risk-score for at least one of recurrence free survival, progression free survival, event free survival, overall survival, response to therapy, or disease-free survival. In some embodiments, a graphical user interface may be communicatively coupled to the at least one server and configured to display a portion of the outcome set.

### BRIEF DESCRIPTION OF THE DRAWINGS

The embodiments described herein will more fully understood from the following detailed description taken in conjunction with the accompanying drawings. The drawings are not intended to be drawn to scale. For the purposes of clarity, not every component may be labeled in every drawing. In the drawings:

FIG. 1 is a block diagram for a system for an artificial intelligence based pathology platform in accordance with some embodiments of the present disclosure;

FIG. 2 is a flow chart for a method for an artificial intelligence based pathology platform in accordance with some embodiments of the present disclosure;

FIG. 3 is a block diagram for a computer system for an artificial intelligence based pathology platform in accordance with some embodiments of the present disclosure;

FIG. 4 is a diagram for a system for an artificial intelligence based pathology platform in accordance with some embodiments of the present disclosure;

FIG. 5 is a diagram for experimental results for an artificial intelligence based pathology platform in accordance with some embodiments of the present disclosure;

FIG. 6 is a diagram for experimental results for an artificial intelligence based pathology platform in accordance with some embodiments of the present disclosure;

FIG. 7A is a diagram for experimental results for an artificial intelligence based pathology platform in accordance with some embodiments of the present disclosure; and

FIG. 7B is a diagram for experimental results for an artificial intelligence based pathology platform in accordance with some embodiments of the present disclosure.

### DETAILED DESCRIPTION

Embodiments of the present disclosure are directed towards systems and methods for predicting outcomes related to cancers. Examples of outcomes include recurrence free survival, progression free survival, event free survival, overall survival, disease-free survival. In some embodiments, a deep learning module is trained on a collection of histological data received from a population of patients having cancer tumors as well as their response to treatments and recurrence of cancer rates. As one example, the disclosed systems and methods may be applied to patients having non-muscle invasive bladder cancer who may be treated with intravesical Bacillus Calmette-Guerin (BCG) therapy. Although applications related to bladder cancer are discussed herein, it is envisioned that applications related to other cancers may utilize similar approaches to those described herein.

The trained deep learning module can be applied to histological samples from a cancer tumor of a patient in order to predict recurrence of cancer for a given treatment or therapy. For example, the deep learning module can be applied to a histological sample for a cancer tumor for a patient, in order to determine a feature set for the histological sample.

In some examples, the deep learning module includes one or more processes for determining the feature set. For example, the deep learning module first determines locations of tissue within the histological sample using threshold based techniques. After detecting areas of tissue cells, the deep learning module applies a U-net architecture to detect positions of nuclei and cells of interest within the identified locations of tissue. In some examples, the U-net architecture is composed of a plurality of convolutional layers configured to first distinguish between objects and background, determine segments of interest within an image based on determined boundaries of the objects, and classify objects. In some embodiments, U-Net model comprises a fully convolutional neural network having an encoder and decoder. The deep learning module may also determine morphologic, geometric, and textural features for each of the detected nuclei and cells of interest. For example, the nuclei may be classified into classes such as Neoplastic, Connective, No-Neoplastic/Epithelial, Necrotic, and Inflammatory. Additionally, the deep learning module may be configured to determine a spatial location feature for each of the detected nuclei and cells of interest based on their correlation and/or overlap. Once a feature set is obtained, the disclosed systems and methods may generate an outcome set for a patient by applying a second multivariate model (e.g., Cox proportional hazards model). The outcome set may be used to predict the likelihood of recurrence/progression/survival/treatment response of cancer in the patient and guide treatment choices by clinicians. The outcomes set can be a risk score on the outcomes or risk categories like high/low risk of recurrence.

For example, the artificial intelligence based pathology platform described herein may provide clinicians with an adjunctive tool that classifies a patient population into “high-risk” or “low-risk” for one or more possible outcomes. This classification may occur at the time of disease diagnosis based on an analysis of a histological sample. Further, the disclosed artificial intelligence based pathology platform may leverage existing workflow and standards of care by using histological samples and other data that is routinely collected and provide clinicians with prognostic information regarding recurrence risk for a cancer for a given therapy prior to the initiation of a particular therapy.

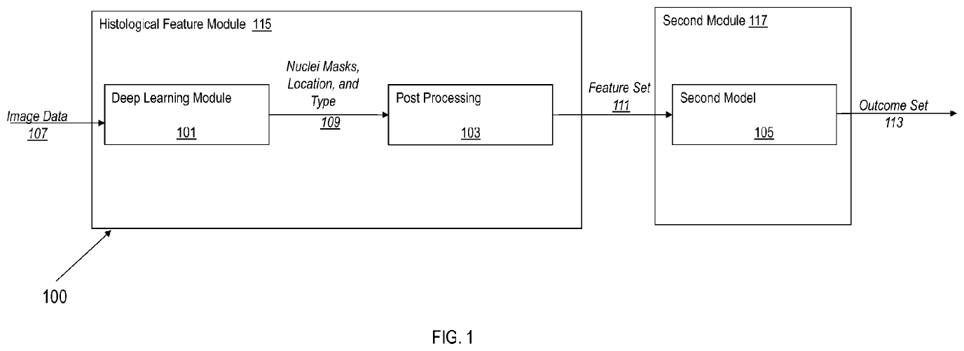

FIG. 1 shows a block diagram for an example of an artificial intelligence based pathology platform 100 for predicting patient outcomes related to cancer. The platform 100 includes a histological feature module 115 that includes a deep learning module 101 and one or more post processing algorithms 103. The platform 100 also includes a second module 117 which includes one or more algorithmic models. For example, a second model 105 may be included in the second module 117. Image data 107 is provided to a deep learning module 101 that is configured to output nuclei location and shape data 109. In some embodiments, post processing algorithms 103 are applied to the nuclei location and shape data 109 in order to generate a feature set 111. The feature set 111 are input into a second model 105 that produces an outcome set 113.

Image data 107 may include digital pathology slides of cancer specimens. For example, this may include whole slide images (WSI) or virtual microscopy images which are digital scans of samples (e.g., tissue sections). WSI or virtual microscopy images may allow for the digitalization of glass slide images. In some embodiments, the image data 107 may be stained using hematoxylin and eosin stains (H&E stains). In some embodiments, the image data 107 may include a 256×256 input image of a histopathology slide. In some embodiments, the image data 107 may be stained using immunohistochemistry (IHC) techniques.

In some examples, the image data 107 corresponds to slides taken in connection with a bladder cancer, non-muscle invasive bladder cancer, muscle invasive bladder cancer, urothelial carcinoma of the bladder, squamous cell carcinoma of the bladder, adenocarcinoma of the bladder, or small cell carcinoma of the bladder.

In some embodiments the deep learning module 101 includes a nuclei segmentation and classification algorithm. In some embodiments, the deep learning module 101 utilizes CellCS. In some embodiments, the deep learning module 101 utilizes a U-Net architecture. The nuclei segmentation and classification approach (CellCS) may form the deep learning model that uses the U-Net architecture as its basic element.

The deep learning module 101 may be configured to distinguish between objects of interest and background, perform segmentation, and classify identified nuclei into respective cell classes. For example, the deep learning module 101 may include three independent convolutional layers of size 3×3×128 that are applied to the output of the final layer of the U-Net model to respectively predict pixel-level (i) normalized object instance probabilities to distinguish between objects of interest and the background, (ii) 32-ray radial distances to boundaries of objects for segmentation, and (iii) cell class probabilities for the classification of nuclei into any number of cell classes.

The first of the three independent convolutional layers may form the object instance layer, configured to help distinguish between objects of interest and background. For a given input, the object instance layer may predict normalized scores for each pixel in the input region to identify if that pixel is associated with a nuclei region or the background.

The second of the three independent convolutional layers may form the segmentation layer, which is configured to determine boundaries of objects for segmentation. In particular, for each pixel in some embodiments 32 radial distance values are predicted to identify the edge of the predicted segmentation for a pixel if that pixel was part of an object.

The third of the three independent convolutional layers may form the cell classification layer. The cell classification layer may compute cell class probabilities corresponding to the likelihood a given cell is of a particular cell class. In some embodiments, the cell classification layer is composed of an n+1 channel predicted mask where a single channel mask corresponds to normalized predictions for each pixel corresponding to a certain cell class on the patch. The classes consist of n cell classes and a background class.

Examples of cell classes may include five, fourteen, or any other number of different classes. In some embodiments, the five classes may include Neoplastic, Connective, No-Neoplastic/Epithelial, Necrotic or Inflammatory. In some embodiments, the fourteen classes may be Neoplastic, Lymphocyte, Macrophage, Non-neoplastic, plasma cell, eosinophils, neutrophils, fibroblasts, erthrocytes, endothelial, muscle, myoepithelium, adipocytes, apoptosis, and mitosis.

In some embodiments, the deep learning module 101 may refine predictions of a single object across multiple pixels using non-maximal suppression above a given object threshold. The majority class probability prediction across the entire object is used to classify the object. For a given segmentation mask, a set of all segmentation masks that were suppressed using non-maximal suppression are used to refine the pixel level segmentation of the object.

In some embodiments, the segments may undergo a shape refinement procedure applied by the deep learning module 101. In a shape refinement procedure, all polygons for an object instance are rasterized as binary masks and aggregated by majority vote in order to obtain the mask of an object instance.

The deep learning module 101 may be evaluated and validated on the basis of its model loss. In some embodiments, model loss is composed of three separate components: a distance regression component, a probability map component and a classification component. The separate components may be aggregated with a weighted sum to form the complete model loss. For example, the distance regression component may correspond to the clipped absolute difference between the 32 distance predictions at each pixel locations. The clipped absolute difference may be weighted by the corresponding ground truth probability for that pixel location and the resulting tensor is then average pooled and normalized by the mean value of the ground truth probability map to produce a scalar corresponding to the distance regression loss.

The probability map component may correspond to the average pooled binary cross-entropy between the predicted and ground truth probability map.

The classification component may correspond to the average pooled cross entropy between the predicted probability of each type per pixel with the class-map.

Together, the final model loss for the deep learning model can be characterized as follows:

$$Model Loss = \{ Weight Factor \} \times \{ Distance Regression \} + \{ Probability Map Component \} + \{ Classification Component \}$$

In some examples, the ground truths include an instance mask with a class map connecting instance indices to class indices. The Euclidean Distance Transform is applied to a binarized mask of nucleus or background pixel level classifications to generate a ground truth probability map. The 32 radial distances to the boundaries of objects are generated at each pixel location to create the distance map. A class map image is generated with each pixel equal to the class index if it is part of a nucleus or zero if it is not.

In some embodiments, the deep learning module 101 is trained using training data that includes annotations of cell segmentation and classification data that is annotated from patches extracted from histopathology images and slides. The training data may include marked cell centroids for all cells in the patches along with the classification of the cells in the region. The cell classes may include 5, 14, or any number of different classes. In some embodiments, the five classes may include Neoplastic, Connective, No-Neoplastic/Epithelial, Necrotic or Inflammatory.

In some embodiments, the deep learning module 101 applies tissue segmentation, nuclei segmentation and geometric feature extraction to the image data 107. In some embodiment, the deep learning module 101 may receive image data 107 that is pre-processed. Examples of pre-processing of the image data 107 include excluding background regions of a whole slide image. In some embodiments, excluding background regions may involve applying color-based thresholding using the lightness channel of the CIELAB color space that was binarized using Otsu's method. For example, in some embodiments, image data 107 may be preprocessed using a single intensity threshold to separate pixels within the received image data 107 into foreground or background. Further, in some embodiments, pre-processing may include identifying patches of appropriate size that would be provided to the deep learning module 101. For example, in some embodiments patches of size 2132×2132 (533×533 μm) are extracted from tissue regions. Pre-processing may also include one or more processes for detecting and removing artifacts.

As discussed above, the deep learning module 101 may then be used to segment and classify each nucleus automatically into a class (e.g., Neoplastic, Connective, No-Neoplastic/Epithelial, Necrotic, Inflammatory). Further, the deep learning module 101 may perform geometric feature extraction on the resulting classified nuclei. For example, the centroids, bounding boxes, and contours of the nuclei may be calculated. The geometric feature extraction may result in shape data.

In some examples, the deep learning module 101 is configured to output nuclei location and shape data 109. In some embodiments the histological feature module 115 may include one or more computer vision techniques to provide nuclei location and shape data 109.

A post processing algorithm 103 may be applied to the output of deep learning module 101 in order to generate a feature set 111. The feature set 111 may include morphology data, tissue region data, spatial relationship data, colocalization data, and hotspot data. The feature set 111 may be computed from the geometric features extracted from the classified nuclei. For example, the feature set 111 may be computed from centroids and contours for each nuclei.

The feature set 111 may include morphology data. Morphology data may be descriptive of morphometric features of nuclei. For example, morphology data may include information about the dimensions, perimeter, area, curvature and eccentricity of nuclei. Morphology data may be computed using segmentation masks, and provide characterizations of the area surrounding nuclei. For example, the morphology data may indicate areas of neoplastic nuclei.

The feature set 111 may include tissue region data. Tissue region data may classify tissue regions according to the maximum cell type proportion predicted by the deep learning module 101. For example, the centroids of nuclei determined by geometrical extraction by the deep learning module 101 may be used to create a spatial mesh using the Delaunay triangulation algorithm. Measurements of the area and perimeter of the triangles formed by the mesh may then be calculated. Examples of features that provide tissue region data include features related to identifying an area of tumor, an area of stroma, and the density of tumor.

The feature set 111 may also include spatial relationship data. The spatial relationship data may indicate relationships between nuclei and cells. For example, spatial relationship data may include spatial statistics of nucleus centroids, nucleus features, and triangle features of different types which may be calculated globally at a slide level and locally in sub-regions (variable sized regions in the slide). Examples of the spatial relationship data may also include colocalization metrics, spatial correlations between features, Moran's indices, measures of spatial entropy, and total variance.

One example of spatial relationship data is colocalization data. Colocalization may be computed on regions within the whole slide image. In some embodiments, colocalization data may be indicative of correlations of the counts of cells between multiple cell types. For example, the counts of cells may be computed on regions within the regions (sub-regions). The counts of cells in the sub-regions may be correlated across the region to compute the colocalization of the corresponding region. In some embodiments, the correlation may be computed by two different metrics: Pearson's correlation coefficient (PCC) and Mander's overlap coefficient (MOC). The sub-regions and the regions can be variable sized sections. Examples of spatial relationship data that may be included in a feature set include data regarding the colocalization of neoplastic and immune cells.

Another example of spatial relationship data is hotspot data. In some embodiments, a spatially connected set of regions within a whole slide image may be defined as a super-region. A super-region that meets a certain pre-defined specification may be considered a hotspot. For example, hotspot features may include the shapes, sizes, counts, and areas of the hotspot. In another example, hotspot data may reveal whether the number of nuclei in a particular super-region having a nuclear area exceeds a threshold amount.

In some embodiments, one or more sub-features may be generated for each sub-region in a whole slide image. Sub-features may correspond to, but are not limited to, the shape and size of a cell, hues of a group of pixels, and the like. Features may be computed as an aggregation of sub-features, such that the features correspond to regions in the whole slide image. Each region may be composed of a set of sub-regions. Similarly, a super-region may be a collection or set of regions and a corresponding super-feature can be computed based on the features of the regions within the super-region. In some embodiments, hotspots may be defined as a super-region whose super-feature meets a defined threshold. A hotspot feature may be a geometrical feature of the hotspot itself.

In some embodiments, morphology data, tissue region data, spatial relationship data, colocalization data, and hotspot data may be aggregated across the whole slide image. For example, each of the morphology data, tissue region data, spatial relationship data, colocalization data and hotspot data may be computed for each sub-region within a whole slide image and then aggregated across the whole slide image with measures like mean, median, standard deviation, interquartile ranges and multiple percentile values (e.g., 5, 10, 15, 25 . . . 75, 85, 95, 99). Examples of features that may be aggregated include data indicating a 95th percentile neoplastic nuclear area.

In some embodiments, morphology data, tissue region data, spatial relationship data, colocalization data, and hotspot data may be aggregated to produce a final feature vector for the whole slide image.

The post processing 103 module of the histological feature module 115 may include one or more algorithms configured to intake the nuclei location and shape data 109 (e.g., nuclei masks, location and type) and output a feature set 111, including the morphology data, tissue region data, spatial relationship data, colocalization data, and hotspot data.

Algorithms included in the post processing 103 module may include those configured for determining morphologic, geometric, textural features of nuclei and/or cells. These may include algorithms for fitting ellipses, bounding boxes, algorithms for calculating the area of morphologic features, algorithms for calculating the hue and staining features. In some embodiments, additional artificial intelligence based models that are trained to calculate features within defined regions using supervised or unsupervised learning may be used.

The feature set may be input into a second module 117 which may include one or more additional algorithmic models. For example, the second module 117 may include second model 105 that is configured to produce an outcome set 113. In some embodiments the second model 105 may include a multivariate model. In some embodiments the second model 105 may include a Cox proportional hazards (CPH) model.

In some embodiments, the second module 117 may sub-select features to form a feature set that is strongly associated with the outcome of interest in the population. In order to do so, the histologic assay may be normalized on the training dataset by subtracting the mean and dividing by the standard deviation and then applying this transformation onto the test dataset. Features can then be pruned by training independent univariate cox proportional hazards models on the training set. Features that have a high concordance index on the training set may then be selected. In order to prevent overfitting towards any specific dataset, features that are associated with histopathological features that have been previously identified in the clinical literature may be subselected. For example, in some applications related to bladder cancer, the features in the histological assay that were associated with the outcome of interest were geometric features of the extent of stromal invasion in neoplastic regions and the variation of morphology of neoplastic cells in inflammatory regions.

In some embodiments the second model 105 may include a multivariate cox proportional hazards model with the sub selected features associated with the outcome of interest being trained on the entire training set. The second model 105 may be trained on a feature set based on a population of histological samples and known outcomes.

After training, the module may generate weights which will determine how features from the feature set are to be combined. These combination of weights and associated features may create a signature. By applying the signature to incoming feature set, the model may generate a risk category or risk score. The risk score or category can then predict an outcome such as recurrence or progression. For example, in some embodiments, a percentile threshold may be used to categorize “low” and “high” risk categories. For example, the 50th percentile response of the predicted expected lifetimes of data points in the training set was used to set the cut-off threshold between “low” and “high” risk categories.

In some embodiments, the second model 105 may also be trained on non-histological data including at least one of medical images, clinical variables, genomics, and medical text.

In some embodiments the second model 105 may generate an outcome set that includes at least one of a risk category, or risk-score for at least one of recurrence free survival, progression free survival, event free survival, overall survival, response to therapy, or disease-free survival. Recurrence free survival may refer to the length of time after primary treatment for a cancer ends that the patient survives without any signs or symptoms of that cancer. Progression free survival may refer to the length of time during and after the treatment of a disease, such as cancer, that a patient lives with the disease but it does not get worse. Event free survival may refer to the length of time after primary treatment for a cancer ends that the patient remains free of certain complications or events that the treatment was intended to prevent or delay. Overall survival may refer to the percentage of people in a study or treatment group who are still alive for a certain period of time after they were diagnosed with or started treatment for a disease, such as cancer. Response to therapy may respond to clinical observations such as tumor shrinkage, tumor death, and the like. Disease-free survival may refer to the length of time after primary treatment for a cancer ends that the patient survives without any signs or symptoms of that cancer.

Based on the outcome set, in some embodiments, the artificial intelligence platform may be configured to produce a graphical user interface for a clinician, printed reports, an indication for an electronic health record, and the like. In some embodiments, a clinician may be able to decide on a course of treatment based on the outcome set. In some embodiments, the platform 100 may be further configured to generate and provide a set of recommended therapies responsive to the determined feature set for the histological sample. For example, the platform 100 may be trained with histological samples and outcome data for a plurality of treatment options and provide recommendations for selecting a treatment option based on the histological features of a sample. In some embodiments, a graphical user interface may display at least a portion of the outcome set.

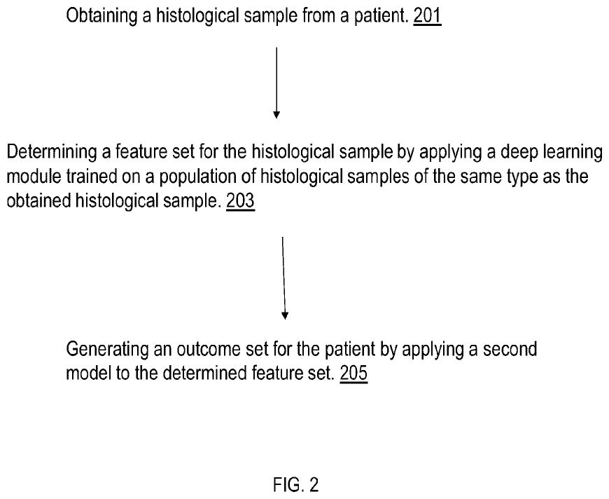

FIG. 2 illustrates a flowchart for a method built in accordance with some embodiments of the present disclosure. A method for predicting outcomes related to a cancer may include the step of obtaining a histological sample of a cancer tumor of a patient 201. In a second step, the method may determine a feature set for the histological sample by applying a deep learning module trained on a population of histological samples of cancer tumors of the same type as the obtained histological sample of the cancer tumor 203. In a third step, a method may generate an outcome set for the patient by applying a second model to the determined feature set 205.

As discussed above, the method may utilize components of the artificial intelligence pathology platform illustrated in FIG. 1. Accordingly, the method may also include training the deep learning module 101 on a population of histological samples of cancer tumors of the same type as the obtained histological sample of the cancer tumor to determine nuclei location and shape data 109.

Further, the method may include determining a feature set for the histological sample by determining locations of tissue within the histological sample, detecting positions of nuclei and cells of interest within the determined locations of tissue, determining at least one of morphologic, geometric, and textural features for each of the detected nuclei and cells of interest, and determining a spatial location feature for each of the detected nuclei and cells of interest.

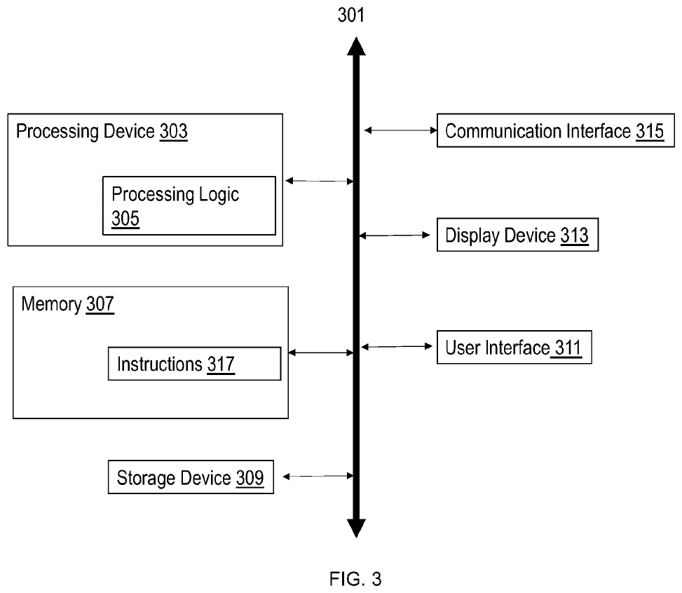

FIG. 3 illustrates a functional block diagram of a machine in the example form of computer system 300, within which a set of instructions for causing the machine to perform any one or more of the methodologies, processes or functions discussed herein may be executed. In some examples, the machine may be connected (e.g., networked) to other machines as described above. The machine may operate in the capacity of a server or a client machine in a client-server network environment, or as a peer machine in a peer-to-peer (or distributed) network environment. The machine may be any special-purpose machine capable of executing a set of instructions (sequential or otherwise) that specify actions to be taken by that machine for performing the functions describe herein. Further, while only a single machine is illustrated, the term “machine” shall also be taken to include any collection of machines that individually or jointly execute a set (or multiple sets) of instructions to perform any one or more of the methodologies discussed herein. In some examples, the platform 100 of FIG. 1 may be implemented by the example machine shown in FIG. 3 (or a combination of two or more of such machines).

Example computer system 300 may include processing device 303, memory 307, data storage device 309 and communication interface 315, which may communicate with each other via data and control bus 301. In some examples, computer system 300 may also include display device 313 and/or user interface 311. In some embodiments, the user interface 311 may include a graphical user interface.

Processing device 303 may include, without being limited to, a microprocessor, a central processing unit, an application specific integrated circuit (ASIC), a field programmable gate array (FPGA), a digital signal processor (DSP) and/or a network processor. Processing device 301 may be configured to execute processing logic 305 for performing the operations described herein. In general, processing device 303 may include any suitable special-purpose processing device specially programmed with processing logic 305 to perform the operations described herein.

Memory 307 may include, for example, without being limited to, at least one of a read-only memory (ROM), a random access memory (RAM), a flash memory, a dynamic RAM (DRAM) and a static RAM (SRAM), storing computer-readable instructions 317 executable by processing device 303. In general, memory 307 may include any suitable non-transitory computer readable storage medium storing computer-readable instructions 317 executable by processing device 303 for performing the operations described herein. Although one memory device 307 is illustrated in FIG. 3, in some examples, computer system 300 may include two or more memory devices (e.g., dynamic memory and static memory).

Computer system 300 may include communication interface device 315, for direct communication with other computers (including wired and/or wireless communication), and/or for communication with a network. In some examples, computer system 300 may include display device 313 (e.g., a liquid crystal display (LCD), a touch sensitive display, etc.). In some examples, computer system 300 may include user interface 311 (e.g., an alphanumeric input device, a cursor control device, etc.).

In some examples, computer system 300 may include data storage device 309 storing instructions (e.g., software) for performing any one or more of the functions described herein. Data storage device 309 may include any suitable non-transitory computer-readable storage medium, including, without being limited to, solid-state memories, optical media and magnetic media.

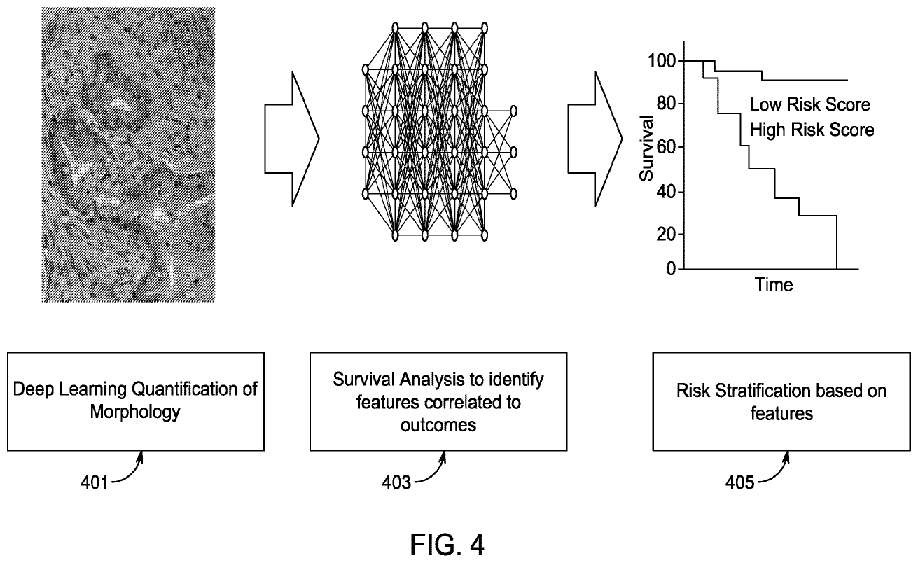

FIG. 4 is a diagram for a system for an artificial intelligence based pathology platform in accordance with some embodiments of the present disclosure. As illustrated in FIG. 4 and discussed herein, in a first step the artificial intelligence based pathology platform may apply deep learning to quantify morphology 401. Then in a second step, the pathology platform may apply survival analysis to identify features correlated to outcomes 403. And finally, in a third step, the pathology platform may apply risk stratification to identify patients having high or low risk scores based on the identified features 405.

### Example: Non-Muscle Invasive Bladder Cancer (NMIBC)

Non-muscle invasive bladder cancer (NMIBC) represents approximately 70% of bladder cancers and encompasses a wide spectrum of disease behavior and resultant patient outcomes. Responses to intravesical Bacillus Calmette-Guérin (BCG) therapy among patients with non-muscle invasive bladder cancer (NMIBC) remain heterogeneous.

Existing clinical risk stratification tools are sometimes intended to quantify the risk of recurrence and/or progression and include those developed by the American Urologic Association (AUA), the European Organisation for Research and Treatment of Cancer (EORTC), Club Urologico Espanol de Tratamiento Oncologico (CUETO), and the European Association of Urology (EUA). However, these tools have been found to be suboptimal due to fundamental limitations in their design, which often lead to an overestimate the risk of recurrence and progression of cancer, including for those NMIBC patients receiving intravesical BCG therapy.

An artificial intelligence platform built in accordance with the description herein was used to analyze digitized whole slide image (WSI) histologic sections derived from pre-treatment transurethral resection of bladder tumor (TURBT) specimens to stratify risk of recurrence in high risk NMIBC patients treated with BCG. The association of the histological assay stratification to recurrence free survival (RFS), BCG response and 12 month recurrence rates at a single institution was evaluated.

### Data

A retrospective study of BCG-treated high-risk NMIBC patients treated at a single institution, the University of Texas Medical Branch at Galveston (UTMB) from January 2014 to December 2021 provided training and validation data. Patients in the study who did not receive adequate BCG therapy (i.e., 5/7 doses within 12 months), those who could not be assessed for BCG-unresponsive disease (per FDA standards), and/or those lacking follow-up at least 1 year post-BCG completion were excluded from the data set. A board-certified genitourinary pathologist selected a representative Hematoxylin and Eosin (H&E) diagnostic slide for each patient obtained by TURBT.

### Model Training

To construct the histological assay, a deep-learning module first segmented nuclei from digital WSIs of the H&E specimens to extract quantitative histological features. These features were then correlated to RFS on the training set (33 patients) utilizing a multivariate Cox proportional hazards (CPH) model. RFS stratification was examined using Kaplan-Meier analysis and log-rank test on the test set (35 patients). Prognostic value of the histological assay was assessed using a multivariate CPH model with available clinical features.

The deep learning module, analogous to model 101 of FIG. 1, was trained for 400 epochs using the Adam optimizer with a learning rate of 0.0003. During training time, geometric augmentations (flips, 90 degree rotations) and brightness augmentations were applied to each input image. To prevent overfitting, the model weights which had the lowest score on the validation dataset were used. The deep learning model was then evaluated on the held out test dataset, achieving DICE scores of 0.77, 0.59, 0.68, 0.24, 0.62 in Neoplastic, Connective, No-Neoplastic/Epithelial, Necrotic, Inflammatory classes, respectively.

Further, the deep learning module, analogous to model 101 of FIG. 1 was trained with annotations of cell segmentation and classification data annotated from patches extracted from histopathology images of bladder cancer slides. For example, 1048×1048 patches at a 40× magnification were extracted from trans-urethral bladder resection tumors (TURBT) slides across (X, Y, Z) sites. Training data (number of patches: 170) was generated by a generalist annotator who marked cell centroids for all cells in these regions with 5 classes (Neoplastic, Connective, No-Neoplastic/Epithelial, Necrotic, Inflammatory).

A second model, analogous to model 105 of FIG. 1, was trained on feature sets and identified outcomes from the data. The 50th percentile response of the predicted expected lifetimes of data points in the training set was used to set the cut-off threshold between “low” and “high” risk categories. Outcome stratification of the model was examined using Kaplan-Meier analysis, log-rank test and c-index on the test set. Recurrence rate was compared across low and high risk categories generated by the risk assessment model.

### Application of the Models

Scanned histologic images were analyzed through an imaging pipeline that included tissue segmentation, nuclei segmentation, and finally geometric feature extraction. Tissue was segmented via color-based thresholding to remove empty regions of the slide. Patches of size 2132×2132 were extracted from tissue regions and a validated deep learning model was used to segment and classify each nucleus automatically into five classes (i.e., Neoplastic, Connective, No-Neoplastic/Epithelial, Necrotic, Inflammatory). Descriptive morphometric features were then computed for each nucleus. Geometric features were then aggregated first at the patch, and subsequently at the patient level using summary statistics including the mean, standard deviation, skewness and kurtosis to produce the final feature vector for a patient. This feature vector was used as the input to a cox proportional hazards model that used the least absolute shrinkage and selection operator to identify the most correlated features with RFS along with their coefficients on the training set.

### Results

A total of 68 patients were included with a median follow-up of 18 months. The “low risk” group as classified by the histological assay had superior RFS compared with the “high risk” group with a Hazard Ratio (HR) of 12.5, Confidence Interval (CI): 1.56-100, log-rank: p=0.003). 10 of 20 patients classified as “high risk” had recurrence events during follow-up compared to 1 of 15 patients classified as “low risk” having recurrence events. Recurrence rates at 12 months were 43.7% in the high risk group and 0% in the low risk group. 8 of 9 BCG unresponsive patients in the test set were classified in the high risk group and one was classified in the low risk group. The histological assay was prognostic independent of CIS and T-stage of the initial TURBT (p<0.05). The sensitivity and specificity of the assay's prediction of recurrence and BCG unresponsiveness were 90%, 58% and 88%, 45% respectively.

### Platform Evaluation

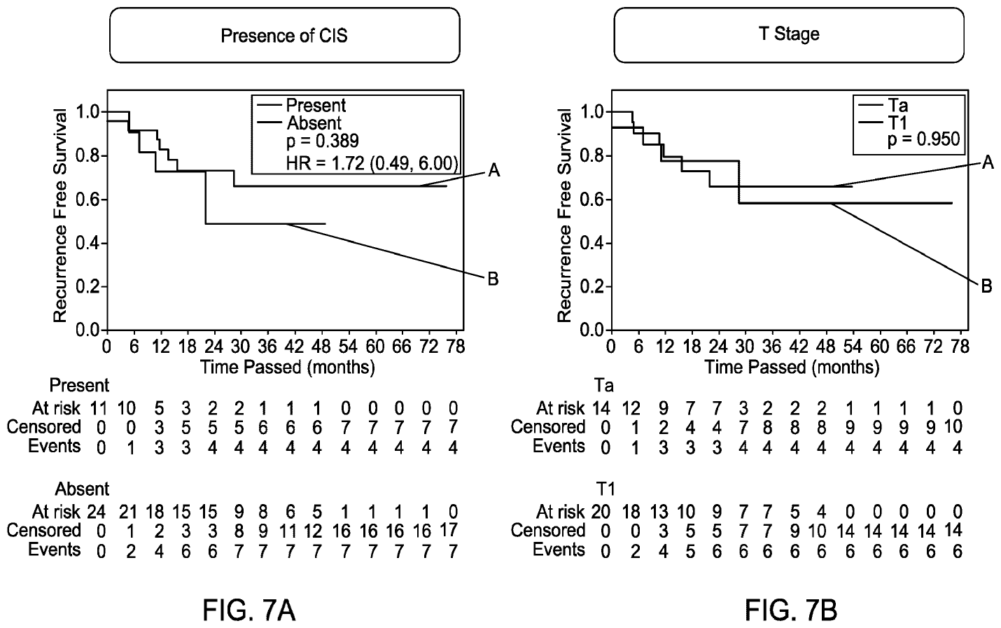

Further, as illustrated in Table 1 and FIGS. 7A and 7B, the prognostic value of risk assessment model applied by the platform described herein was assessed by comparing output of the risk assessment model when a multivariate Cox proportional hazards model was used to multivariate Cox proportional hazards models based on conventional clinical markers such as the presence of carcinoma in situ (CIS) and T-stage of the initial TURBT (i.e., Ta indicating non-invasive papillary carcinoma, or T1 indicating tumor spread to connective tissue). Accordingly, an artificial intelligence-based platform utilizing pre-treatment H&E stained histopathology specimens may further assist in the identification of patients with high-risk NMIBC most likely to recur following BCG therapy.

TABLE 1

| | | | Recurrence Rate | | BCG | Histology of Initial TURBT | | |
|---|---|---|---|---|---|---|---|---|
| Risk | | Recurrence | (months) | | unresponsive | Presence of | | |
| Stratification | Patients | Events | 12 | 18 | events | CIS | Ta | T1 |
| Test | 35 | 11 | 23.3% | 33.3% | 9 | 11 | 14 | 21 |
| Population | | | | | | | | |
| Predicted | 20 | 10 | 43.7% | 53.3% | 8 | 8 | 7 | 13 |
| High Risk | | | | | | | | |
| Predicted | 15 | 1 | 0% | 8.3% | 1 | 3 | 7 | 8 |
| Low Risk | | | | | | | | |

As illustrated in Table 1, the test population and data for the disclosed example included 35 patients of which 11 had recurrence events. Further, 9 patients were unresponsive to the BCG treatment plan. As illustrated in Row 2, the artificial intelligence based pathology platform predicted 20 of the 35 patients as being high risk. As illustrated in Row 3, the artificial intelligence based pathology platform predicted 15 of the 35 patients as being low risk. As demonstrated in the recurrence events column, 10 of the 20 patients predicted to be high risk had a recurrence event. Similarly, only 1 of the 15 patients predicted to be low risk had a recurrence event. The table further provides histological information about the presence of clinical markers such as CIS and T-stage (i.e., Ta and T1) that are often used to determine whether a patient is high or low risk in conventional systems. As shown in Table 1, the presence of clinical markers such as CIS and T-stage (i.e., Ta and T1) are not definitively indicative of the likelihood of a patient having a recurrence event.

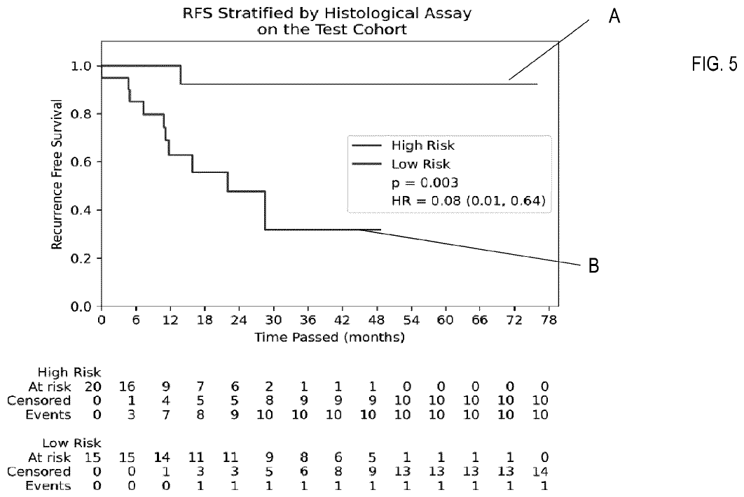

FIG. 5 is a diagram for experimental results for an artificial intelligence based pathology platform in accordance with some embodiments of the present disclosure. In particular, FIG. 5 provides a Kaplan Meier plot for the risk stratification of recurrence free survival as provided by an artificial intelligence based pathology platform built in accordance with the disclosure herein. As demonstrated, the platform categorized a first group A as being low risk and a second group B as high risk. As illustrated in the predicted high risk group 20 percent of patients have a recurrence event within 6 months. By contrast, in the predicted low risk group only 10 percent of patients have a recurrence event at 24 months.

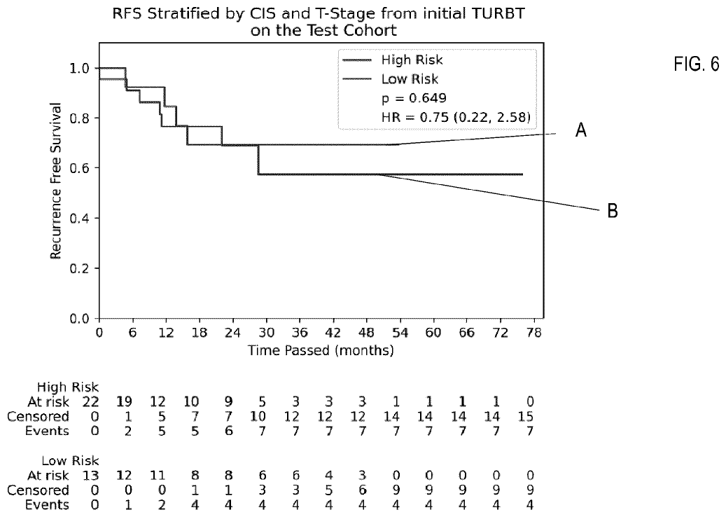

FIG. 6 is a diagram for experimental results for an artificial intelligence based pathology platform in accordance with some embodiments of the present disclosure. In particular FIG. 6 demonstrates how well existing risk stratifications based on presence of carcinoma in situ (CIS) and T-stage of the initial TURBT compare. As demonstrated, the conventional metrics based on CIS and T-stage categorized a first group A as being low risk and a second group B as high risk.

FIGS. 7A and 7B demonstrate how existing clinical variables perform at stratifying high risk patients. In particular FIG. 7A provides data utilizing presence of CIS (B) or absence of CIS (A) as means to characterize recurrence free survival. Additionally, FIG. 7B provides data utilizing T-Stage, specifically Ta (B) or T1 (A) as a means to characterize recurrence free survival.

One skilled in the art will appreciate further features and advantages of the invention based on the above-described embodiments. Accordingly, the invention is not be limited by what has been particularly shown and described, except as indicated by the appended claims.

1. A method performed by at least one processor for predicting outcomes related to a cancer, the method comprising:
obtaining a histological sample of a cancer tumor of a patient;
determining a feature set for the histological sample by applying a deep learning module trained on a population of histological samples of cancer tumors of the same type as the obtained histological sample of the cancer tumor; and
generating an outcome set for the patient by applying a second model to the determined feature set.

2. The method of claim 1, wherein the outcome set comprises at least one of a risk category, or risk-score for at least one of recurrence free survival, progression free survival, event free survival, overall survival, response to therapy, or disease-free survival.

3. The method of claim 1, wherein the cancer is at least one of bladder cancer, non-muscle invasive bladder cancer, muscle invasive bladder cancer, urothelial carcinoma of the bladder, squamous cell carcinoma of the bladder, adenocarcinoma of the bladder, and small cell carcinoma of the bladder.

4. The method of claim 1, further comprising:
providing a set of recommended therapies responsive to the determined feature set for the histological sample.

5. The method of claim 1, wherein the feature set for the histological sample comprises at least one of morphology data, tissue region data, spatial relationship data, colocalization data, and hotspot data.

6. The method of claim 1, wherein the deep learning module comprises a U-Net model, wherein the U-Net model comprises a fully convolutional neural network having an encoder and decoder.

7. The method of claim 1, further comprising:
training the deep learning module on the population of histological samples of cancer tumors of the same type as the obtained histological sample of the cancer tumor to determine nuclei location and shape data.

8. The method of claim 1, wherein determining a feature set for the histological sample further comprises:
determining locations of tissue within the histological sample;
detecting positions of nuclei and cells of interest within the determined locations of tissue;
determining at least one of morphologic, geometric, and textural features for each of the detected nuclei and cells of interest; and
determining a spatial location feature for each of the detected nuclei and cells of interest.

9. The method of claim 1, wherein the second model comprises a multivariate model.

10. The method of claim 9, wherein the multivariate model comprises a Cox proportional hazards (CPH) model.

11. The method of claim 1, further comprising:
training the second model on non-histological data comprising at least one of medical images, clinical variables, genomics, and medical text.

12. The method of claim 1, further comprising:
training the second model to determine a signature, wherein the signature comprises the combination of histological features and weights.

13. The method of claim 1, further comprising:
administering to the patient a particular treatment type, responsive to the outcome set corresponding to the particular treatment type.

14. The method of claim 1, further comprising:
displaying, on a graphical user interface, at least a portion of the outcome set.

15. A non-transitory computer-readable medium storing instructions that, when executed on one or more processors, cause the one or more processors to:
obtain a histological sample of a cancer tumor of a patient;
determine a feature set for the histological sample by applying a deep learning module trained on a population of histological samples of cancer tumors of the same type as the obtained histological sample of the cancer tumor; and
generate an outcome set for the patient by applying a second model to the determined feature set.

16. The non-transitory computer-readable medium of claim 15, wherein the instructions further include instructions that cause the one or more processors to:
display, on a graphical user interface, at least a portion of the outcome set.

17. The non-transitory computer-readable medium of claim 15, wherein the instructions further include instructions that cause the one or more processors to determine the feature set for the histological sample by determining locations of tissue within the histological sample, detecting positions of nuclei and cells of interest within the determined locations of tissue, determining at least one of morphologic, geometric, and textural features for each of the detected nuclei and cells of interest, or determining a spatial location feature for each of the detected nuclei and cells of interest.

18. The non-transitory computer-readable medium of claim 15, wherein the second model comprises a multivariate model.

19. A system for predicting outcomes related to a cancer, the system comprising:
at least one server communicatively coupled to a user device by a network, wherein the at least one server further comprises a non-transitory memory storing computer-readable instructions and at least one processor;
the execution of the computer-readable instructions causing the at least one server to:
train a deep learning module on a population of histological samples of cancer tumors, wherein the deep learning module comprises a U-net model;
train a second model on feature set data and outcomes data, wherein the second model comprises a multivariate model;
obtain a histological sample of a cancer tumor of a patient, wherein the cancer tumor is of the same type as the population of histological samples of cancer tumors;
determine a feature set for the histological sample by applying the trained deep learning module; and
generate an outcome set for the patient by applying the trained multivariate model to the determined feature set.

20. The system of claim 19, wherein the feature set comprises at least one of morphology data, tissue region data, spatial relationship data, colocalization data, and hotspot data.

21. The system of claim 19, wherein determining the feature set comprises the execution of computer-readable instructions causing the at least one server to:
determine locations of tissue within the histological sample;
detect positions of nuclei and cells of interest within the determined locations of tissue;
determine at least one of morphologic, geometric, and textural features for each of the detected nuclei and cells of interest; or
determine a spatial location feature for each of the detected nuclei and cells of interest.

22. The system of claim 19, wherein the outcome set comprises at least one of a risk category, or risk-score for at least one of recurrence free survival, progression free survival, event free survival, overall survival, response to therapy, or disease-free survival.

23. The system of claim 19, further comprising a graphical user interface, communicatively coupled to the at least one server, wherein the graphical user interface is configured to display a portion of the outcome set.

24. The method of claim 1, wherein the histological sample comprises a whole slide image and/or virtual microscopy image.

25. The method of claim 8, further comprising:
determining a cell type for the detected cells of interest, wherein the cell type comprises a tumor cell, immune cell, or stromal cell.

26. The method of claim 25, wherein the cell type comprises at least one of neutrophil, lymphocyte, eosinophil, tumor/neoplastic, macrophage, mitosis, plasma, endothelial, apoptosis or stromal.

27. The method of claim 13, wherein administering to the patient the particular treatment type, responsive to the outcome set for a particular treatment type comprises determining at least one of a risk category, or risk-score for at least one of recurrence free survival, progression free survival, event free survival, overall survival, response to therapy, or disease-free survival corresponding to a particular treatment type.

28. The non-transitory computer-readable medium of claim 15, wherein the histological sample comprises a whole slide image and/or virtual microscopy image.

29. The non-transitory computer-readable medium of claim 17, further comprising:
determining a cell type for the detected cells of interest, wherein the cell type comprises a tumor cell, immune cell, or stromal cell.

30. The non-transitory computer-readable medium of claim 29, wherein the cell type comprises at least one of neutrophil, lymphocyte, eosinophil, tumor/neoplastic, macrophage, mitosis, plasma, endothelial, apoptosis or stromal.

31. The non-transitory computer-readable medium of claim 15, further comprising instructions for:
administering to the patient a particular treatment type, responsive to the outcome set for a particular treatment type indicating at least one of a risk category, or risk-score for at least one of recurrence free survival, progression free survival, event free survival, overall survival, response to therapy, or disease-free survival corresponding to a particular treatment type.

32. The system of claim 19, wherein the histological sample comprise a whole slide image and/or virtual microscopy image.

33. The system of claim 19, wherein execution of computer-readable instructions causes the at least one server to:
determine a cell type for the detected cells of interest, wherein the cell type comprises a tumor cell, immune cell, or stromal cell.

34. The system of claim 33, wherein the cell type comprises at least one of neutrophil, lymphocyte, eosinophil, tumor/neoplastic, macrophage, mitosis, plasma, endothelial, apoptosis or stromal.

35. The system of claim 19, wherein the execution of computer-readable instructions causes the at least one server to:
administer to the patient a particular treatment type, responsive to the outcome set for a particular treatment type indicating at least one of a risk category, or risk-score for at least one of recurrence free survival, progression free survival, event free survival, overall survival, response to therapy, or disease-free survival corresponding to a particular treatment type.
